(19) United States
(12) Patent Application Publication
(10) Pub. No.: US 2004/0242556 A1
(43) Pub. Date: Dec. 2, 2004

(54) Novel crystalline form of cefdinir

(76) Inventors: Ramesh Dandala, Hyderabad (IN); Meenakshisunderam Sivakumaran, Hyderabad (IN)

Correspondence Address: Jay R Akhave, 845 Pomello Dr, Claremont, CA, 91711, US

(21) Appl. No.: 10/634,978

(22) Filed: Feb. 24, 2004

(30) Foreign Application Priority Data
Jun. 2, 2003 (IN) 440/MAS/2003

(51) Int. Cl. A61K 31/549

(52) U.S. Cl. 514/202; 544/220

(57) ABSTRACT

The present invention relates to novel crystalline form of Cefdinir, 7β-[(Z)-2-(2-amino-4-thiazolyl)-2-hydroxyiminoacetamido]-3-vinyl-3-cephem-4-carboxylic acid, herein called as cefdinir crystal B, process to prepare it and the use of cefdinir crystal B in pharmaceutical compositions.

### FIELD OF INVENTION

A new inventive polymorph of crystalline Cefdinir of Formula I, a process of making the same and using it in a pharmaceutical composition.

### BACKGROUND OF THE INVENTION

Cefdinir of Formula I is a very useful antimicrobial agent and is chemically known as 7β-[(Z)-2-(2-amino-4-thiazolyl)-2-hydroxyiminoacetamido]-3-vinyl-3-cephem-4-carboxylic acid.

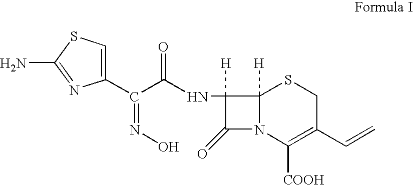

Cefdinir is an oral antibiotic and was first disclosed in U.S. Pat. No. 4,559,334. The product obtained according to above invention was crystalline like amorphous product, not a crystalline product.

U.S. Pat. No. 4,935,507 discloses the methods of producing crystalline cefdinir that offered better filtration rate, high purity and stable cefdinir suitable for pharmaceutical preparation. This was prepared by treating amorphous cefdinir with sodium bicarbonate solution and the resulting aqueous solution was subjected to column chromatography and then adjusting the pH between 1-2 at 35-40° C. followed by cooling to obtain cefdinir crystals A. Alternatively, amorphous cefdinir was dissolved in methanol and to this solution added water at 35° C., stirred and allowed to stand at room temperature to obtain cefdinir crystals A.

Though, U.S. Pat. No. 4,935,507 claims crystalline form A, advantages may yet be realized by others, heretofore undiscovered forms of cefdinir. The present invention includes a novel crystalline form of cefdinir. Polymorphism is the property of some molecules and molecular complexes to assume more than one crystalline or amorphous form in solid state. A molecule like cefdinir of Formula I may give to a variety of solids having distinct physical properties like solubility, melting point, powdered X-ray diffractogram (XRD). The differences in the physical properties of polymorphs result from the orientation and intermolecular interactions of adjacent molecules (complexes) in the bulk solid. Accordingly, polymorphs are distinct solids sharing the same molecular formula, which may be thought of as analogous to unit cell metallurgy, yet having distinct advantageous and or disadvantageous physical properties compared to other forms in the polymorph family. The present invention provides a new crystalline form of cefdinir having moisture content in the range of 5.5-7.0%, but typically 6-7%, amounting closely to sesquihydrate and herein called as cefdinir crystal B. Further, the major advantages realized in the present invention are avoiding the use of chromatographic technique and highly pure cefdinir crystal B is obtained. It is an objective of the present invention to provide a stable, nontoxic, non-solvate crystalline modification of cefdinir, which is substantially free of impurities.

### BRIEF DESCRIPTION OF ACCOMPANYING DRAWINGS

FIG. 1 is a characteristic X-ray powder diffractogram of cefdinir crystal B

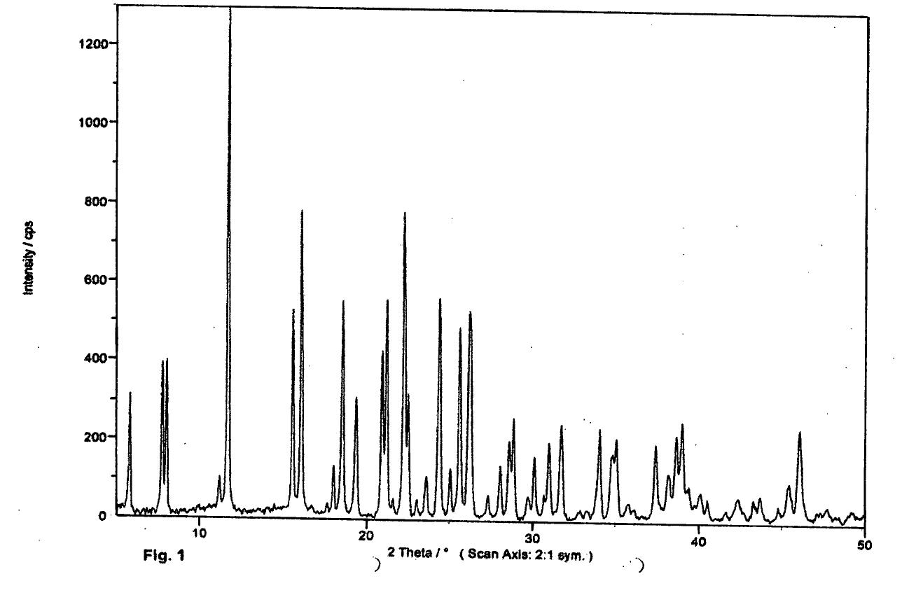

Vertical axis: Intensity (CPS)

Horizontal axis: Two theta (degrees)

The significant d values (2θ) obtained are:

5.8±0.2, 11.7±0.2, 16.1±0.2, 18.6±0.2, 20.9±0.2, 22.2±0.2,

24.4±0.2, 25.6±0.2 etc.

FIG. 2 is a X-ray powder diffractogram of cefdinir crystal A

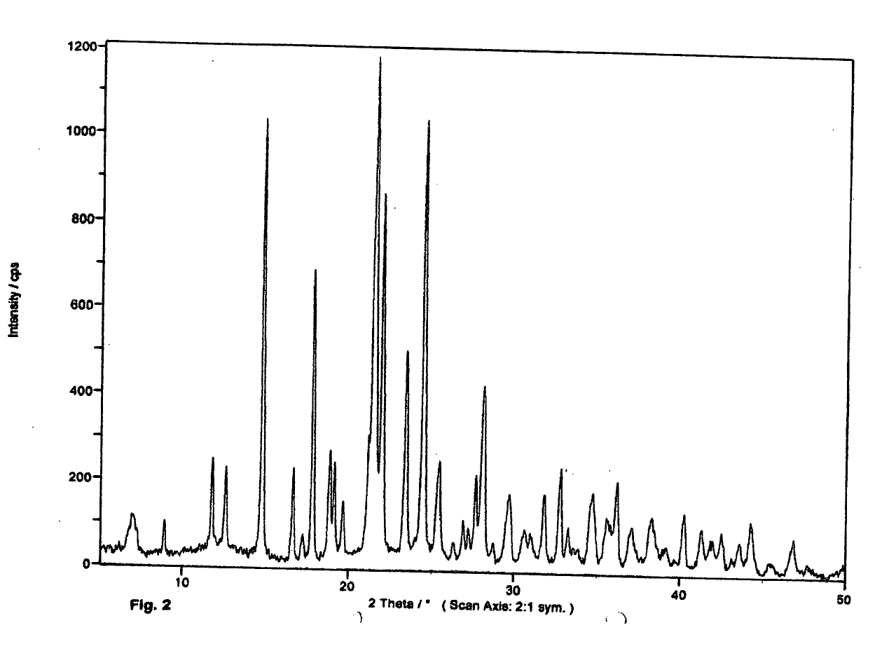

Vertical axis: Intensity (CPS)

Horizontal axis: Two theta (degrees).

The significant d value (2θ) obtained are

11.7±0.2, 14.7±0.2, 17.8±0.2, 21.5±0.2,

21.9±0.2, 23.4±0.2, 24.5±0.2, 25.4±0.2 etc.

FIG. 3 is the infrared absorption spectrum of cefdinir crystal B.

FIG. 4 is the infrared absorption spectrum of cefdinir crystal A.

### DETAILED DESCRIPTION OF THE INVENTION

The present invention relates to a novel crystalline form of cefdinir, hereinafter called as cefdinir crystal B. More particularly, the novel form may contain water up to 5.5-7.0% by weight but typically close to 6.3% w/w which corresponds to the stoichiometric value of ˜1.5 mole of water per mole of cefdinir. The novel crystal B of cefdinir of the present invention may be characterized by powdered X-ray diffraction and infrared absorption spectrum. Thus powdered X-ray diffraction of novel cefdinir crystal B and crystal A were determined on Seifert XRD 3003TT system using a copper target X-ray tube, a nickel filter and the sample was placed in a pyrex glass holder. The scan rate was 1.8 degrees, two theta per minute with the step size of 0.03 degrees two theta over the range from 5 to 50 degrees.

The novel cefdinir crystal B has powdered X-ray diffraction pattern essentially as shown in Table 1 and the powdered X-ray diffraction pattern reported for crystal A is given in Table 2. The powdered X-ray diffraction patterns are expressed in terms of two theta and relative intensities.

TABLE-1
LIST OF 2θ AND RELATIVE INTENSITY FOR
CEFDINIR CRYSTAL B.

| 2 θ | RELATIVE INTENSITY (%) |
|---|---|
| 5.8 | 25 |
| 7.7 | 31 |
| 8.0 | 31 |
| 11.7 | 100 |
| 15.6 | 42 |
| 16.1 | 61 |
| 18.6 | 44 |
| 19.4 | 25 |
| 21.0 | 34 |
| 21.2 | 44 |
| 22.3 | 61 |
| 24.4 | 44 |
| 25.6 | 38 |

TABLE-2
LIST OF 2θ AND RELATIVE INSENTIES FOR
CEFDINIR CRYSTAL A

| 2 θ | RELATIVE INTENSITY (%) |
|---|---|
| 6.9 | 9 |
| 8.8 | 8 |
| 11.7 | 21 |
| 12.5 | 19 |
| 14.7 | 89 |
| 16.5 | 20 |
| 17.8 | 59 |
| 18.8 | 22 |
| 21.5 | 100 |
| 21.9 | 73 |
| 23.4 | 42 |
| 24.5 | 87 |
| 25.4 | 20 |

The infrared absorption spectra of cefdinir crystal B and crystal A were determined on Perkin Elmer-spectrum ONE infrared spectrophotometer.

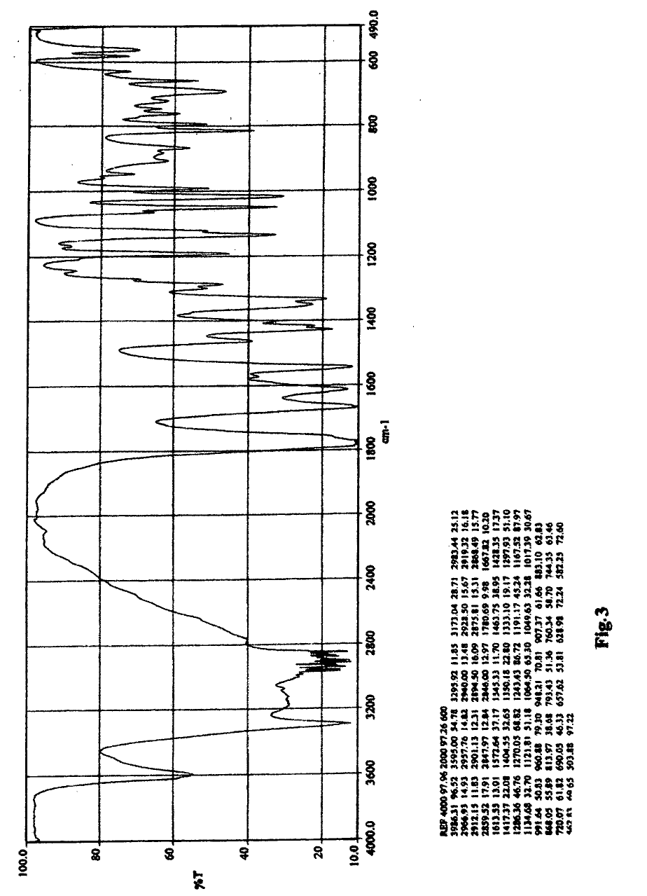

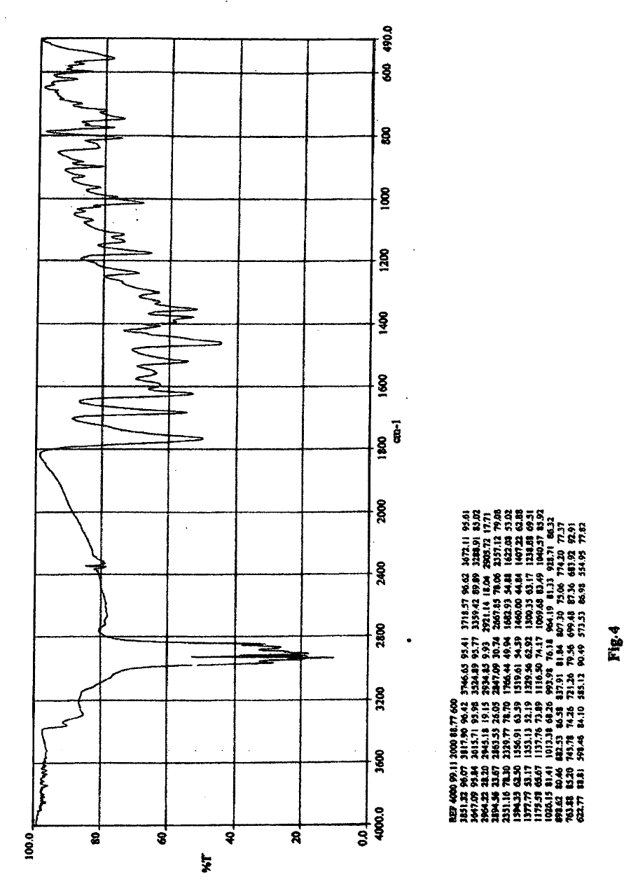

The infrared absorption spectrum of cefdinir crystal B (FIG. 3) shows characteristics peaks at 1017, 1049, 1121, 1134, 1191, 1428, 1545, 1613, 1667, 1780, 3295and 3595 Cm⁻¹.

Whereas the infrared spectrum of cefdinir crystal A (FIG. 4) shows characteristics peaks at 1013, 1175, 1460, 1519, 1556, 1594, 1622,1682 and 1766 Cm⁻¹.

It is evident from the above data that the novel crystalline form of present invention is different from the cefdinir crystal A reported in U.S. Pat. No. 4,935,507. The powdered X-ray diffraction pattern of crystal B shows maximum peak at 11.7±0.2 degree two theta whereas crystal A shows maximum peak at 21.5±0.2 degree two theta. We have found that this novel cefdinir crystal B has excellent storage stability characteristics and suitable for pharmaceutical formulation.

The stress stability studies of cefdinir crystal B were carried out. The samples were kept under the conditions of 60° C.±2° C. and the purity and assay of samples were determined before and after the stress test by high performance liquid chromatography and given in Table 3 below.

TABLE-3
STRESS STABILITY DATA OF CEFDINIR CRYSTAL B

| | |
|---|---|
| Purity (by HPLC) | Assay (by HPLC) |

| Sample | Initial | After stress | Initial | After stress |
|---|---|---|---|---|
| 1 | 99.66% | 99.45% | 99.9% | 100.75% |
| 2 | 99.71% | 99.38% | 99.4% | 99.52% |

As shown in the Table-3 even after keeping the samples at 60° C.±2° C. for fifteen days the compounds did not degrade. Therefore, it is evident that cefdinir crystal B is quite stable.

The present invention also relates to the process for the preparation of novel cefdinir crystal B.

The process comprises the step of condensation of 7-amino-3-vinyl-3-cephem4-carboxylate 4-methoxybenzyl ester hydrochloride with 2-benzothiazolyl (Z)-2-(2-amino-4-thiazolyl)-2-trityloxyiminothioacetate in the presence of trialkylamine in any suitable solvent such as N,N-dimethylacetamide, N,N-dimethylformamide, methylene dichloride and like and mixture thereof. Specifically the condensation is carried out at a temperature range of 40-50° C. After completion of reaction, the reaction mass is cooled and product is extracted into suitable organic solvent and washed with dilute base and water. The suitable solvent can be selected from methylene dichloride, chloroform, toluene, ethylene dichloride etc., most preferably methylene dichloride. Further, organic layer is treated with trifluoroacetic acid at a temperature of about 10-15° C. for 4-5 h to remove carboxyl protecting groups. Thereafter, the organic layer was cooled to 0° C. and water was added. The layers were separated, and on cooling the aqueous layer, trifluoroacetic acid salt of cefdinir precipitated out, which is isolated by filtration.

Further, the wet cefdinir trifluoroacetic acid salt is suspended in water and neutralized with aqueous ammonia at a temperature of 0-30° C. most preferably at 20-25° C. to obtain highly purified cefdinir crystal B.

A further aspect of the present invention is a process to produce cefdinir crystal B from cefdinir crystal A.

The process comprises of suspending cefdinir crystal A in water at 35-40° C. and treating with trifluoroacetic acid to prepare cefdinir.trifluoroacetic acid salt. The trifluoroacetic acid salt of cefdinir is isolated in high purity. Typically purity is analyzed by HPLC and is greater than 99.5% and generally closer to 99.6%. The trifluoroacetic acid salt of cefdinir is then neutralized by adjusting the pH to 3.0-3.2 with aqueous ammonia in water at 0-30° C. preferably at 20-25° C. to obtain highly pure cefdinir crystal B as off white solid. Typically, the purities are greater than 99% by HPLC in commercial lots. The trifluoroacetic acid salt of cefdinir is preferably used as wet material without drying.

The cefdinir crystal A can be prepared by methods known in the art (U.S. Pat. No. 4,935,507) and then can easily be converted to cefdinir crystal B.

The present invention hence provides a novel crystalline form, crystal B of cefdinir and a method of its preparation, which is amenable to large-scale production, and suitable for formulation.

The novel crystalline form, crystal B of cefdinir of the present invention is readily filterable and easily dried. Moreover, the cefdinir crystal B prepared is of high purity typically greater than 99.5% as seen by HPLC. The novel crystalline form shows excellent storage stability and hence suitable for formulation. The novel crystal B of cefdinir may contain 5.5-7.0% of water and has a decomposition range of 188-192° C.

Having thus described the various aspects of the present invention, the following examples are provided to illustrate specific embodiments of the present invention. They are not intended to be limiting in anyway.

### EXAMPLE 1

### PREPARATION OF 7β-[(Z)-2-AMINO-4-THIAZOLYL)-2-HYDROXYIMINO ACETAMIDO]-3-VINYL-3-CEPHEM-4-CARBOXYLIC ACID. TRIFLUOROACETIC ACID SALT (CEFDINIR. TRIFLUOROACETIC ACID SALT)

50 g of 7-Amino-3-vinyl-3-cephem-4-carboxylate-4-methoxybenzyl ester hydrochloride (0.130 mol) and 71.5 g of 2-benzothiazolyl (Z)-2-(2-amino-4-thiazolyl)-2-trityloxyiminothioacetate (0.124 mol) were suspended in 250 ml of N,N-dimethylacetamide and 12.5 g of triethylamine (0.124 mol) was added thereto. Then the mixture was stirred for 3 hours at 40-50° C. After cooling to 10° C., methylene dichloride (750 ml) was added followed by demineralised water (1000 ml) and stirred for 10 minutes at the same temperature. Layers were separated and the organic layer was washed with dilute sodium hydroxide solution and water respectively. The methylene dichloride layer was cooled to 10° C. Trifluoroacetic acid (300 ml) was added over a period of 30 minutes at 10-15° C. and stirred for 4 hours at the same temperature. Thereafter, cooled the reaction solution to 0° C. Demineralised water (875 ml) was added and separated the layers. Aqueous extract was cooled to 0° C. and stirred for 60 minutes. The precipitate thus obtained was filtered and washed with ice-cooled water (125 ml) to obtain trifluoroacetic acid salt of cefdinir as an off white crystalline solid having purity 99.5% by HPLC.

¹H-NMR inDMSO-d₆: δ(ppm); 3.58 and 3.83 (Abq, 2H, J=17.84), 5.20 (d, 1H, J=4.94 Hz), 5.32 (d, 1H, J=11.25 Hz), 5.60 (d, 1H, J=17.56 Hz), 5.79 (dd, 1H, J=4.94 Hz and 8.23 Hz), 6.76 (s, 1H), 6.92 (dd, 1H, J=11.25 Hz and 17.56 Hz), 9.60 (d, 1H, J=7.96 Hz)

### EXAMPLE 2

### PREPARATION OF 7β-[(Z)-2-(2-AMINO-4-THIAZOLYL)-2-HYDROXYIMINO ACETAMIDO]-3-VINYL-3-CEPHEM-4-CARBOXYLIC ACID (CEFDINIR CRYSTAL B)

The wet product 70β-[(Z)-2-(2-amino-4-thiazolyl)-2-hydroxyiminoacetamido]-3-vinyl-3-cephem-4-carboxylic acid.trifluoroacetic acid salt obtained in Example-1 was suspended in water (875 ml) and cooled to 10° C. pH was adjusted to 3.0-3.2 with aqueous ammonia solution at 20-25° C. and filtered, washed with water (250 ml) and thus dried to obtain 24 g of cefdinir crystal B as a off white solid. (HPLC Purity: 99.6%).

Water content (% w/w, by KF): 7.0%; Melting point: 190° C. (decompose).

¹H-NMR in DMSO-d6: δ(ppm); 3.55 and 3.84 (Abq, 2H, J=17.56 Hz), 5.19 (d, 1H, J=4.94 Hz), 5.31 (d, 1H, J=11.25 Hz), 5.59 (d, 1H, J=17.56 Hz), 5.79 (dd, 1H, J=4.94 Hz and 8.23 Hz), 6.66(s, 1H), 6.90 (dd, 1H, J=11.25 Hz and 17.56 Hz), 7.13 (bs, 2H), 9.48 (d, 1H, J=8.23 Hz).

### EXAMPLE 3

### PREPARATION OF 7β-[(Z)-2-(2-AMINO-4-THIAZOLYL)-2-HYDROXYIMINO ACETAMIDO]-3-VINYL-3-CEPHEM-4-CARBOXYLIC ACID (CEFDINIR CRYSTAL B)

10 g of Cefdinir crystal A was suspended in 300 ml of demineralised water at 30-35° C. 30 ml of trifluoroacetic acid was added in 15 minutes at 30-35° C. to get a clear solution. After about 15-20 minutes, precipitation was started and stirred for 60 minutes at 40° C. Precipitate thus obtained, was filtered, washed with chilled water (20 ml) to obtain trifluoroacetic acid salt of cefdinir. This salt without further drying was suspended in demineralised water (200 ml) at 20-25° C. and the pH was adjusted to 3.0-3.2 with aqueous ammonia solution at 20-25° C. Stirred the resulting slurry for 30 minutes, filtered, washed with water (50 ml) and dried to get cefdinir crystal B as an off white solid. (HPLC Purity: 99.6%)

Water content (% w/w, by KF): 6.67%; Melting point: 190° C. (decompose)

¹H-NMR in DMSO-₆: δ(ppm); 3.56 and 3.84 (Abq, 2H, J=17.56 Hz), 5.19 (d 1H, J=4.94 Hz), 5.31 (d, 1H, J=11.25 Hz), 5.59 (d, 1H, J=17.56 Hz), 5.79 (dd, 1H, J=4.94 Hz and 8.23 Hz), 6.67 (s, 1H), 6.91 (dd, 1H, J=11.25 Hz and 17.56 Hz), 7.15 (bs, 2H), 9.50 (d, 1H, J-8.23 Hz).

We claim:

1. A crystalline 7β-[(Z)-2-(2-amino-4-thiazolyl)-2-hydroxyiminoacetamido]-3-vinyl-3-cephem-4-carboxylic acid (Crystal B of Cefdinir) which shows peaks in its powder X-ray diffraction pattern at the diffraction angles of about 5.8±0.2, 11.7±0.2, 16.1±0.2, 18.6±0.2, 20.9±0.2, 22.2±0.2, 24.4±0.2 and 25.6±0.2 two theta degrees.

2. Crystalline substance of claim 1 which is characterized by infrared absorption spectrum pattern having characteristic peaks at approximately 1017, 1049, 1121, 1134, 1191, 1428, 1545, 1613, 1667, 1780, 3295 and 3595 Cm⁻¹.

3. Crystalline substance of claim 1, which contains water in the range of 5.5 to 7.0% by weight.

4. A process for preparing crystalline 7β-[(Z)-2-(2-amino-4-thiazolyl)-2-hydroxyiminoacetamido]-3-vinyl-3-cephem-4-carboxylic acid (Crystal B of cefdinir) which comprises the steps of:
Reacting crystals A of cefdinir in water with trifluoroacetic acid at 35-40° C. to form cefdinir trifluoroacetic acid salt,
optionally isolating the said cefdinir.trifluoroacetic acid salt,
neutralizing the said cefdinir.trifluoroacetic acid salt by treatment with a base in water at a temperature between 0° C. to 30° C., isolating crystal B of cefdinir by filtration.

5. The process according to claim 4, wherein the base used for neutralization is preferably ammonia.

6. The process according to claim 4, wherein, the said neutralization step is conducted at a temperature range of 0-30° C. and preferably at 20-25° C.

7. A pharmaceutical composition comprising a therapeutically effective amount of Crystal B of cefdinir and a pharmaceutically acceptable carrier.
